(19) United States
(12) Patent Application Publication
(10) Pub. No.: US 2016/0279439 A1
(43) Pub. Date: Sep. 29, 2016

(54) METHODS OF APPLICATION OF THERAPEUTIC LIGHT SOURCES

(71) Applicant: Akari Systems, Inc., Sunnyvale, CA (US)

(72) Inventor: Philip Arnold Ferolito, Sunnyvale, CA (US)

(21) Appl. No.: 15/078,839

(22) Filed: Mar. 23, 2016

Related U.S. Application Data
(60) Provisional application No. 62/177,665, filed on Mar. 23, 2015.

(51) Int. Cl. A61N 5/06

(52) U.S. Cl. CPC A61N 5/0616; A61N 2005/0626; A61N 2005/0645; A61N 2005/0652; A61N 2005/0661

(57) ABSTRACT

A light source to provide therapeutic benefits to a patient's skin, with optical elements used to control light incidence on the skin at angles greater than ±20 degrees to the perpendicular to the skin surface, and methods for irradiating the patient's skin with a wearable device providing light at such angles.

### CROSS-REFERENCE TO RELATED APPLICATIONS

This application claims the benefit of U.S. Provisional Application No. 62/177,665 filed Mar. 23, 2015, incorporated by reference in its entirety herein.

### FIELD OF THE INVENTION

The present invention relates generally to a light source with therapeutic benefits including optical elements used to control light incident on the surface of the patient's skin to angles roughly greater than ±30 degrees to the perpendicular to the skin surface.

### BACKGROUND

Adoption of new treatments, especially those associated with long term prevention of disease benefit greatly when they may be administered through an existing activity requiring no behavioral changes to the general population. A fine example of this is the introduction of iodine in table salt in the United States early in the 20^{th} century, effectively eliminating much of the existing thyroid disease without requiring individuals to alter their normal behavior even though the alternative was simply to take a supplement. Additionally toilets have undergone steady transitions from a simple hole in the ground at the beginning of the 20^{th} century to the electronically controlled smart toilets now pervasive in countries such as Japan. These personal newer generation toilets are excellent delivery vehicles for therapeutic light as they allow regular treatments, avoid exposure to face and hands, and require exposure to areas of the body least photo damaged by sunlight.

Many research publications (over 3000 of them in 2013) show beneficial association with low intensity ultraviolet radiation (UVR) skin exposure and a host of chronic health conditions. Unfortunately not everyone lives in a geographic location where sunlight contains the required ultraviolet wavelengths year round, resulting in a seasonal fluctuation of critical molecules/hormones in a person's blood, such as vitamin D. In addition to geographical factors influencing insufficient sunlight exposure, modern lifestyles and working habits prevent the bulk of the population from spending adequate time in the sun during the hours when the required wavelengths are present. For example in Boston during the summer, the time period when sunlight exposure can produce vitamin D is between 10 am and 3 pm, a range of time where most individuals are working indoors. In winter, even the noon sun is not sufficient to synthesize vitamin D in the skin.

It has been established that human skin will convert naturally occurring 7-dehydrocholesterol into pre-vitamin D3, which will then begin a process by which serum 25(OH)D levels are raised. Required light wavelengths are between 280 nm and 320 nm and the most effective rate of conversion has been determined to be around 298 nm. The range from 280 nm to 320 nm is in the UVB (ultraviolet B) part of the spectrum and long-term exposure to the eyes is discouraged as it is responsible for increased risks of cataracts and other ocular damage. Due to the potential for long term eye damage, UV filtering sunglasses are recommended when out in the direct sunlight and it would be considered poor practice to intentionally add the 280 nm through 320 nm wavelength light to indoor lighting. Additionally those skilled in the art know that wavelengths less than 290 nm can do considerable direct and indirect damage to DNA and exposure should be avoided.

In addition to endogenous synthesis of vitamin D, light has been shown to produce hundreds of photo-products in skin. Many of these molecules are highly mobile allowing systemic, whole body, effects despite localized generation. These molecules include but are not limited to the production of cis-urocanic acid, nitric oxide, beta-endorphin and the hormone vitamin D. Each activates different, though possibly sympathetic (interrelated), pathways bringing about positive health benefits. These benefits include but are not limited to: vitamin D regulating calcium absorption, bone mineralization and the overall maintenance of calcium homeostasis which is responsible for skeletal health as well as positive effects on vitamin D receptors in virtually all other systems in the human body; mobilization of nitrite in the skin to form nitric oxide, a molecule reacting with the smooth muscles lining arteries, reducing blood pressure, a known risk factor for heart disease. Production of beta-endorphin can help manage pain by interacting with the brain in a manner similar to an opiate.

Human populations are moving steadily away from equatorial regions, modern lifestyles keep many persons indoors all day, and social customs of sun avoidance all contribute to large populations of humans who no longer have access to natural sources of light capable of producing these molecules.

There is a need for therapeutic light sources suitable for efficient and convenient treatment of human patients, for enabling ultraviolet, blue, red, near infrared, and/or infrared light therapy for the promotion of wellbeing, management of pain, healing of wounds, stimulation of vitamin D synthesis, reduction of inflammation, regulation of immune response, reduction of blood pressure, etc.

### SUMMARY OF THE INVENTION

The invention relates generally to light sources with therapeutic benefits including optical elements used to control light incident on the surface of the patient's skin to angles roughly greater than 20 degrees to the perpendicular to the skin surface (incidence angles to the skin of less than 70 degrees or more than 110 degrees).

According to some embodiments, the light sources are directed toward optical elements that are arranged to disperse the light away from incidence angles to the skin of 70 to 110 degrees. In the preferred embodiment these elements are reflective surfaces but in other embodiments they can be refractive lenses or a combination thereof

According to some embodiments, the light sources are arranged to direct light toward skin with a spreading lens and a filtering material deposited (coated) on the lens to attenuate the light with incident angles to the skin of 70 to 110 degrees. In the preferred embodiment, the coating is a material that is reflective to the wavelengths of the light source, allowing light to re-circulate and reflect back at an angle outside the 70 to 110 degree window, and then allowing it to incident the skin. As an example, for wavelengths near 298 nm, aluminum has high reflectivity, even higher than silver. In other embodiments, some efficiency can be sacrificed by simply using a material that will absorb the light between 70 and 110 degrees, for example most plastics (PMMA included) are opaque to 298 nm and inexpensive.

### BRIEF DESCRIPTION OF THE DRAWINGS

These and other aspects and features of the present invention will become apparent to those ordinarily skilled in the art upon review of the following description of specific embodiments of the invention in conjunction with the accompanying figures, wherein:

FIG. 1 shows a simplified model of skin layers (4303) showing a ray of light (4300) penetrating the epidermis (4301) at a right angle (90 degrees to the surface also described as 0 degrees from the perpendicular) to a depth (4302) reaching the stratum Basal.

FIG. 2 shows the same simplified skin model from FIG. 1, but showing a dispersed set of light rays (4400) penetrating to a lesser depth (4402) as the angle (4406) varies away from the perpendicular to the skin surface.

FIG. 3 shows one element of an LED array (4501) with an added optical element (4503) to take in light (4500) from the LED, producing light (4504) shaped so as to reduce the amount of light entering the skin at or near the perpendicular to the skin surface. This figure shows the results of symmetric methods of light adjustment.

FIG. 4 shows one element of an LED array (4605) with an added optical element (4603) to take in light (4604) from the LED, producing light (4607) shaped so as to reduce the amount of light entering the skin at or near the perpendicular to the skin surface. This diagram shows the results of asymmetric methods of light adjustment.

FIG. 5 shows an array of light emitting diodes intended to illuminate a portion of skin. The diodes (4101) are attached to a substrate (4100) and facing away from the substrate.

FIG. 6a shows a diode element from an LED array (5000) with an added dome lens (5002) that bends the majority of the light (5003) away from the perpendicular to the skin surface.

FIG. 6b shows a diode element from an LED array (5100) with an added dome lens (5102) that bends the majority of the light (5103) away from the perpendicular to the skin surface and the addition of an opaque coating further reducing the light emitted along the perpendicular to the skin surface.

FIG. 6c shows a diode element from an LED array (5200) with folded optics utilizing 2 mirrors (5201) (5202) to alter the angle of the emitted light. If the slope of the mirrors is varied, dispersion can also be achieved to spread the light over a wider area.

FIG. 7 shows light projection on a flat surface for an LED with dispersive dome lens (6001) and LED with dispersive dome lens with center filter (6005). The figure additionally shows the relative energy from 30 to 150 degrees from right angle (6003) and (6007) as measured by a slice through the center of the illumination pattern (6002) and (6006) respectively.

FIG. 8 shows a schematic representation of a control system for a therapeutic light source, according to some embodiments of the present invention.

### DETAILED DESCRIPTION

Embodiments of the present invention will now be described in detail with reference to the drawings, which are provided as illustrative examples of the invention so as to enable those skilled in the art to practice the invention. Notably, the figures and examples below are not meant to limit the scope of the present invention to a single embodiment, but other embodiments are possible by way of interchange of some or all of the described or illustrated elements. Moreover, where certain elements of the present invention can be partially or fully implemented using known components, only those portions of such known components that are necessary for an understanding of the present invention will be described, and detailed descriptions of other portions of such known components will be omitted so as not to obscure the invention. In the present specification, an embodiment showing a singular component should not be considered limiting; rather, the invention is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

For an average adult, it is widely accepted that human skin has a surface area between 1.5-2.0 square meters and an average thickness between 2 mm and 3 mm. Areas of skin exposed to friction, for example the pads on the palm of hands while using tools or the bottom of the foot in contact with shoe or ground while walking/running, will thicken as protection. Additionally, it is know that skin generally thickens with age and that age related factors, for example what is typically known as wrinkling, can effectively thicken the skin as well. Human skin is the largest organ in the body. Working inward from the outside, skin is comprised of many layers including the Stratum Corneum, Stratum Lucidum, Stratum Granulosum, Stratum Spinosum and Stratum Basale. New cells are produced in the Stratum Basale by way of Basal cells and over time these cells are pushed outwards, eventually losing their nuclei and finally sloughing off all together. This process creates protective and sacrificial outer layers.

There is a strong correlation between the wavelength of light and the depth to which it will penetrate the skin. As the wavelength gets longer, light will penetrate to deeper layers. A commonly seen example of this is what happens when a very bright white light source is placed behind a portion of skin This skin can be the fold between thumb and forefinger or the light may be allowed to penetrate completely through a finger. The effect seen is that light emitted from the far side of the skin is decidedly red in color. This is because white light is composed of many wavelengths spanning from red (620 nm) to blue/violet (400 nm). Skin will preferentially absorb the shorter wavelengths (blue/violet) and allow progressively more of the longer wavelengths (yellow, orange, and red) to pass through. The thicker the skin, or in the case of passing a light through an entire appendage such as a finger requiring, the more substantial the short wavelength attenuation.

Each wavelength of light describes a specific amount of energy expressed by a single photon at that wavelength. The relationship between wavelength and photon energy is well known [E=hc/lambda ] where h is Planck's constant, c is the speed of light, and lambda is the wavelength of interest. As the wavelength decreases the single photon energy increases, and as wavelength increases the single photon energy decreases.

Wavelengths associated with vitamin D synthesis are potentially carcinogenic and can cause direct and indirect DNA damage. Direct damage is caused by photons colliding with DNA itself while indirect damage is caused by oxidative stress. Damage may take the form of denaturing a portion of the DNA, single or multiple strand damage, cross linked proteins, or other, and the result may be a non-viable cell, resulting cellular death through apoptosis, or worse a viable mutated cell that continues growing. Some small amount of skin damage is occurring at all times and the goal of any light therapy is to minimize damage while maximizing the beneficial effect. Beneficial effects are the efficient production of the desired molecules, in this case including but not limited to one or more of vitamin D, nitric oxide, cis-urocanic acid, and beta-endorphin.

As skin is comprised of layers of different cells, each layer serves a purpose and each layer has a different sensitivity to light damage. Since cells located close to the surface of the skin contain no nuclei, they are not subject to carcinogenesis or mutagenesis and damage they undergo is short lived as these cells fall away from the body within weeks. In general, damage becomes more dangerous as it reaches deeper in to the skin. Most of the potential for tumorgenesis occurs at or below the basal cell layer, involving basal cells, melanocytes, etc.

In summary, shorter wavelengths have a higher photon energy resulting in a higher likelihood of damaging cells they impact but they will penetrate less deeply in to the skin. Light impacting the surface of the skin at a right angle will penetrate more deeply than light impacting with a different angle.

Traditional light therapy devices emit light from a standing light source that is set at a distance from the target skin. This distance may be several inches to several feet, for example the interior of a tanning booth or the surface of a seasonal affected disorder (SAD) lamp. At these distances the intent is to provide a uniform exposure with no attempt to control the angle at which light is incident upon the skin.

The present invention relates to methods to reduce the amount of light, from one or more light sources, that is incident on skin at or near the perpendicular to the skin surface.

FIG. 1 shows a simplified model of skin layers (4303) showing a ray of light (4300) penetrating the epidermis (4301) at a right angle (90 degrees) to a depth (4302) reaching the stratum basale. Light of wavelength lambda (4300) impacting the skin at or near the perpendicular to the skin surface and passing through the epidermal layers (4303) (4301) has a probability P of reaching a specific depth (4302). This probability is dependent on a number of factors including, but not limited to, the wavelength, skin pigmentation, skin thickness, previous exposure to UV light, age, wrinkling, and hair.

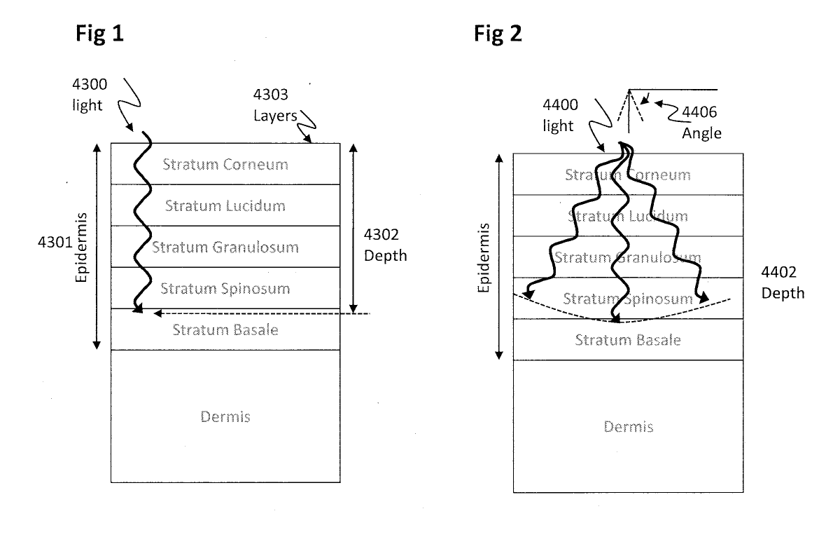

FIG. 2 shows the same simplified skin model from FIG. 1 but showing a dispersed set of light rays (4400) penetrating to a lesser depth (4402) as the angle (4406) varies away from the perpendicular to the skin surface. A therapeutic LED driven device could integrate a spreader or diffuser to create a conical beam (4400) aimed toward the skin. Due to the extreme proximity of the device to the skin the amount of spreading is limited and the depth of penetration (4402) is variable depending on the angle of the incident light. This design still has substantial light energy penetrating to the basal cell layer, a layer which is very active in production of new cells and containing cells best left with minimal damage from UV light.

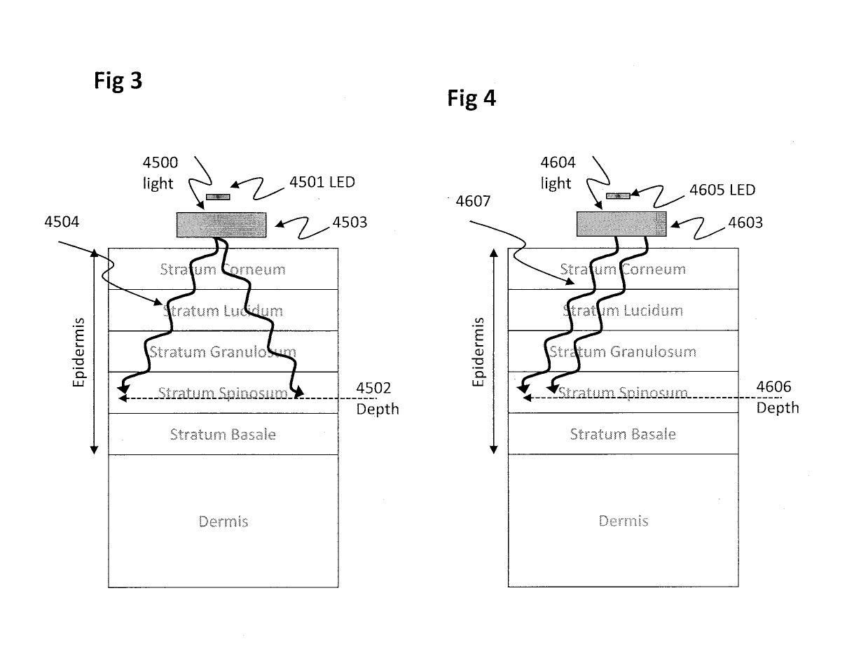

FIG. 3 shows one element of the LED array (4501) with an added optical element (4503) to take in light (4500) from the LED, producing light (4504) shaped so as to reduce the amount of light entering the skin at or near the perpendicular to the skin surface. This diagram shows the results of symmetric methods of light adjustment. Introduction of an optical element between the light source (LED) and the can control the angle at which light is incident to the skin. Using knowledge of the skin penetration and wavelength of light used, the depth can be controlled to limit damage to the more sensitive lower layers of skin. Shown in (4500) one embodiment of this optical element (4503) will take light from a light source (4501) and bend, refract or reflect it so that the majority of light emitted to the skin is at an angle greater than 30 degrees. If the angle is controlled to 45 degrees (for example) the depth of penetration (4502) will be reduced by 30%. Angles greater than 45 degrees reduce the depth of penetration still further but substantial reflection can occur if the angle is allowed to be too shallow.

Furthermore, FIG. 4 shows one element of the LED array (4605) with an added optical element (4603) to take in light (4604) from the LED, producing light (4607) shaped so as to reduce the amount of light entering the skin at or near the perpendicular to the skin surface. This diagram shows the results of asymmetric methods of light adjustment.

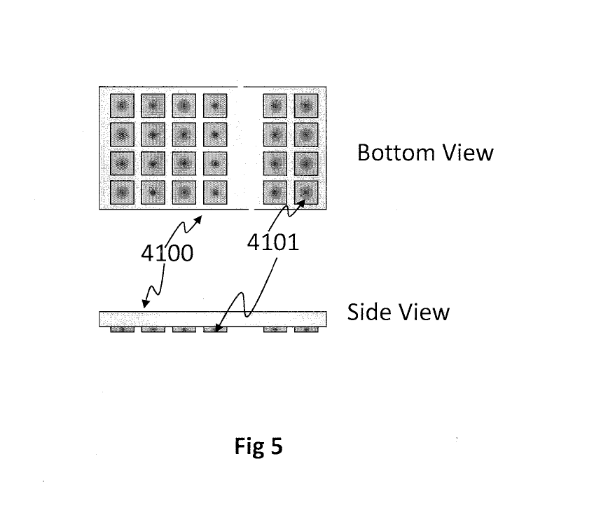

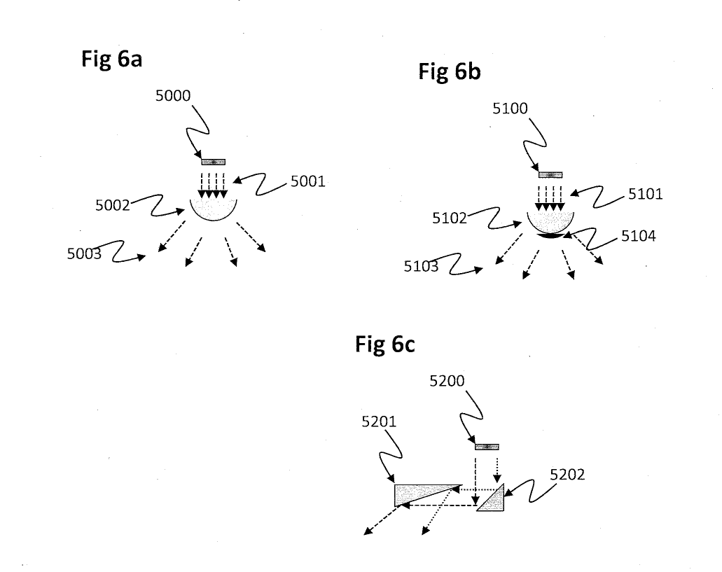

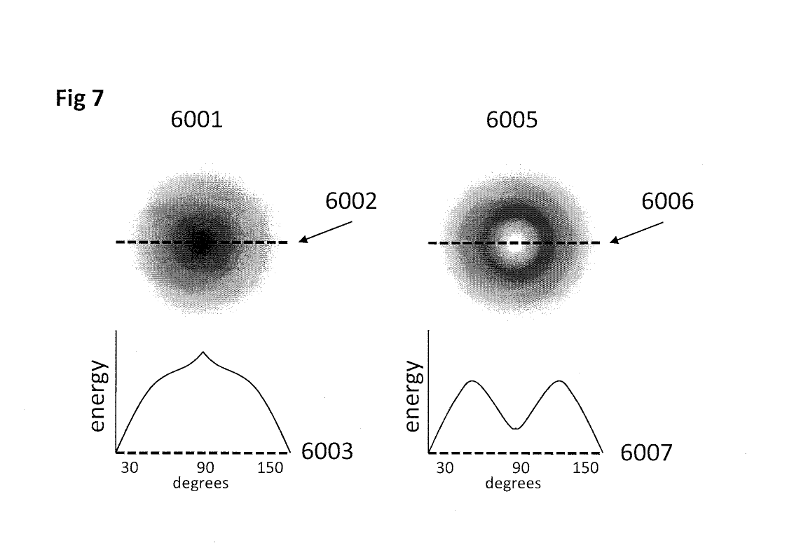

FIG. 5 shows an array of light emitting diodes intended to illuminate a portion of skin. The diodes (4101) are attached to a substrate (4100) and facing away from the substrate. In one embodiment, an array of LEDs (5100) on a substrate can be used with added dome shaped lens (5102) and coating (5104) applied to the center of the lens (FIG. 6b). The coating is applied with near complete opacity at the center and complete transparency at a distance from the center corresponding +or −30 degrees. When this LED side of the substrate is placed close to the skin, close being within 3 cm, the majority of the light energy (5103) incident on the skin is at an angle less than 70 degrees or greater than 110 degrees with peak energy at around 60 degrees and 120 degrees. When compared to the uncoated dispersive element, the depth of penetration in the target skin has been reduced by 15.5% as calculated by well-known geometry. If light of a specific wavelength (300 nm for example) has a probably N to penetrate skin to a depth of 2 mm, then changing the angle from orthogonal to orthogonal +30 degrees reduces the vertical penetration by COS(30)*100=15.4%.

FIG. 6a shows another embodiment with a diode element from the LED array (5000) with an added dome lens (5002) that bends the majority of the light (5003) away from vertical (90 degrees).

FIG. 6c shows an embodiment with a diode element from the LED array (5200) with folded optics utilizing 2 mirrors (5201) (5202) to alter the angle of the emitted light. If the slope of the minors is varied, dispersion can also be achieved to spread the light over a wider area.

FIG. 7 shows the light projection on a flat surface for an LED with dispersive dome lens (6001) and LED with dispersive dome lens with center filter (6005). The figure additionally shows the relative energy from 30 to 150 degrees from right angle (6003) and (6007) as measured by a slice through the center of the illumination patterns (6002) and (6006), respectively. Preferably less than fifty percent of the light is incident to the skin at angles between 70 and 110 degrees, and more preferably less than twenty-five to thirty percent is incident at such angles. Peak energy is preferred at incidence angles of around 55-65 degrees and 115-125 degrees.

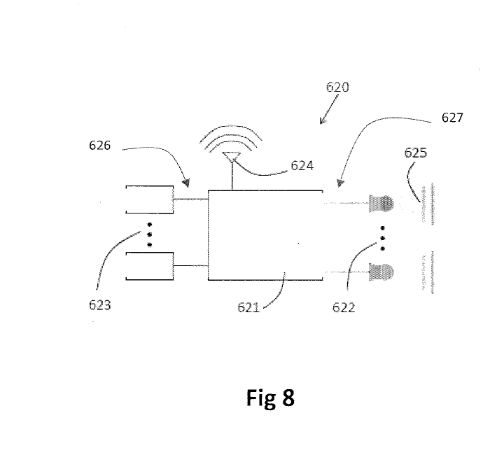

FIG. 8 shows a schematic representation of a control system 620 for a therapeutic light irradiation device, such as a blanket, foldable panels etc., according to some embodiments of the present invention. The control system comprises a controller 621, at least one sensor 623, optional short range wireless antenna 624, and one or more light emitters 622 emitting therapeutic radiation 625. Data from the sensor(s) such as pressure, skin pigmentation and weight can be fed to the controller to enable/disable one or more light emitters and adjust the exposure duration and/or intensity. Furthermore, a power supply for the controller/sensors/light emitters may be a battery, or the mains power may be used. The controller (621) is a device, configurable either through the use of a general purpose instruction set, special purpose microcode, or field programmable gate array. It is capable of receiving information from one or more sensors (623) through one or more electrical interfaces (626) and is able to modify, through other electrical interfaces (627), the amount of light emitted from one or more light sources (622).

Additionally further description of related aspects can be found in the patent application WEARABLE THERAPEUTIC LIGHT SOURCE, filed Jul. 9, 2015 Application No. WO 2016007798 A2, incorporated by reference or in its entirety herein. Therein described are various embodiments of devices and methods of control related to devices including blankets, bands, and pads. The common purpose to these devices is the generation of molecules in skin by means of exposure to light. The common description of these devices includes sensors for detecting device proximity to skin, sensors for detecting pigmentation of skin, an electronically entered profile including skin pigmentation distinguishing at least 4 levels from light to dark, a controller, where the controller is electronically connected to the sensors and light sources with the ability to turn on/off one or more light sources to control the duration of exposure and the controller is able to control the intensity of one or more light sources.

Exposure to UV light is generally measured in independent units of a SED, standard erythemal dose. Each individual, based on exposure history, pigmentation of the skin, age, and other factors has a tolerance measured in a MED. A MED is a minimum erythemal dose, and corresponds to the exposure threshold where skin will react by producing a noticeable pinkening or darkening In general it is desirable to keep to below a 0.5 MED daily exposure to minimize the potential for skin pigmentation changes.

Although wearable devices have been described herein primarily with respect to providing therapeutic UV exposure at wavelengths associated with vitamin D synthesis in humans, the wearable device may also be configured to provide therapeutic exposures: (1) at other wavelengths specifically targeting different conditions or biomarkers, for example IR exposure for the production of nitric oxide, and (2) for other therapeutic effects, for example UV exposure for the treatment of psoriasis. More detailed discussion of the benefits of exposures at various wavelengths is provided in the published PCT application WEARABLE THERAPEUTIC LIGHT SOURCE, filed Jul. 9, 2015 Application No. WO 2016007798 A2, incorporated by reference in its entirety herein. It is envisaged that the embodiments of the wearable devices disclosed herein may be configured to gain the benefit of irradiations of skin at one or more of these wavelengths.

Although embodiments of the present disclosure have been particularly described with reference to certain embodiments thereof, it should be readily apparent to those of ordinary skill in the art that changes and modifications in the form and details may be made without departing from the spirit and scope of the disclosure.

What is claimed is:

1. A light source with therapeutic benefits comprising optical elements used to control light incident on the surface of a patient's skin to angles roughly greater than ±20 degrees to the perpendicular to the skin surface.

2. The light source of claim 1 wherein the light source is an LED array, and the optical elements redirect the light from the LED array so that less than fifty percent of the light energy incident on the skin is at an incidence angle between 70 and 110 degrees to the skin.

3. The light source of claim 2, wherein the peak light energy incident on the skin is at incidence angles around 55 to 65 degrees and around 115 to 125 degrees to the skin.

4. The light source of claim 2, wherein the optical elements are lenses.

5. The light source of claim 4, wherein the lenses attenuate light with incidence angles to the skin of between 60 and 120 degrees.

6. The light source of claim 5, wherein a coating is used to attenuate the light.

7. The light source of claim 6, wherein attenuated light is reflected and redirected by the optical elements to have an incidence angle on the skin of less than 70 degrees or more than 110 degrees.

8. The light source of claim 2, wherein the light source emits light of wavelengths between 290 nm and 310 nm, to promote synthesis of vitamin D.

9. The light source of claim 2, wherein the optical elements are mirrors.

10. A wearable device for therapeutic irradiation of skin, comprising:
a substrate, said substrate having a first surface and a second surface;
a light source comprising an array of light emitting diodes (LEDs) attached to said first surface of said substrate;
optical elements attached to said light source for controlling light incident on the surface of a patient's skin to angles roughly greater than ±20 degrees to the perpendicular to the skin surface;
a controller electrically coupled to said light source, said controller being configured for controlling the intensity of light emitted from said light source and the duration of emission of light from said light source during a therapeutic session;
a proximity sensor for detecting proximity of said substrate to skin, said proximity sensor being attached to at least one of said first surface and said second surface of said substrate, said proximity sensor being electrically coupled to said controller; and
a power source electrically coupled to said light source and said controller;
wherein said controller is further configured to turn on, and keep turned on for said duration of said therapeutic session, said light source when said proximity sensor detects proximity of said light spreading sheet to said skin.

11. The light source of claim 10 wherein the optical elements redirect the light from the LED array so that less than fifty percent of the light energy incident on the skin is at an incidence angle between 70 and 110 degrees to the skin.

12. The light source of claim 11, wherein the peak light energy incident on the skin is at incidence angles around 55 to 65 degrees and around 115 to 125 degrees to the skin.

13. The light source of claim 11, wherein the optical elements are lenses.

14. The light source of claim 13, wherein the lenses attenuate light with incidence angles to the skin of between 60 and 120 degrees.

15. The light source of claim 14, wherein a coating is used to attenuate the light.

16. The light source of claim 15, wherein attenuated light is reflected and redirected by the optical elements to have an incidence angle on the skin of less than 70 degrees or more than 110 degrees.

17. The light source of claim 11, wherein the light source emits light of wavelengths between 290 nm and 310 nm, to promote synthesis of vitamin D.

18. The light source of claim 11, wherein the optical elements are mirrors.

19. A method of irradiating a patient's skin with a wearable device, comprising:
providing a wearable device, said wearable device comprising:
a substrate, said substrate having a first surface and a second surface;
a light source attached to said substrate;
optical elements attached to said light source for controlling light incident on the surface of a patient's skin to angles roughly greater than ±20 degrees to the perpendicular to the skin surface;
a controller electrically coupled to said light source, said controller being configured for controlling the intensity of light emitted from said light source and the duration of emission of light from said light source during a therapeutic session;
a proximity sensor for detecting proximity of said substrate to said patient's skin, said proximity sensor being attached to at least one of said first surface and said second surface of said substrate, said proximity sensor being electrically coupled to said controller; and
a power source electrically coupled to said light source and said controller;
wherein said controller is further configured to only turn on, and keep turned on, said light source when said proximity sensor detects proximity of said substrate to said patient's skin;
placing said wearable device in proximity to said patient's skin;
detecting proximity of said light spreading sheet to said patient's skin by said controller; and
on detecting proximity, turning on said light source by said controller for a prescribed time.
